(19) United States
(12) Patent Application Publication
(10) Pub. No.: US 2023/0111918 A1
(43) Pub. Date: Apr. 13, 2023

(54) Lu-177 RADIOCHEMISTRY SYSTEM AND METHOD

(71) Applicant: BWXT Medical Ltd., Cambridge (CA)

(72) Inventors: Stephen M. OELSNER, Carleton Place (CA); Gary HUNTER, Gatineau (CA); Bryan Blake WIGGINS, Forest, VA (US); Timothy A. POLICKE, Forest, VA (US); David J. GLENN, Forest, VA (US)

(21) Appl. No.: 17/959,752

(22) Filed: Oct. 4, 2022

Related U.S. Application Data
(60) Provisional application No. 63/253,333, filed on Oct. 7, 2021.

(51) Int. Cl. G01N 30/60; G21G 4/08; A61K 41/00

(52) U.S. Cl. CPC G01N 30/6091; A61K 41/009; G21G 4/08

(57) ABSTRACT

A method of making Lu-177 involving dissolving enriched Yb₂O₃, loading dissolved enriched Yb₂O₃ on a first guard column containing resin prepared from (2-ethyl-1-hexyl)phosphonic acid mono(2-ethyl-1-hexyl)ester (HEH[EHP]), passing a first separation of a stream exiting from first guard column through a first resin cartridge containing dipentyl pentylphosphonate, collecting Lu-177 onto a first collection column having resin containing tetraoctyl diglycolamide (DGA), loading an exiting stream from first collection column on a second guard column containing resin prepared from (2-ethyl-1-hexyl)phosphonic acid mono(2-ethyl-1-hexyl)ester (HEH[EHP]); passing a first separation of a stream exiting from second guard column through a second resin cartridge containing dipentyl pentylphosphonate; collecting Lu-177 onto a second collection column having resin containing DGA; passing a second separation of a stream exiting from second guard column through a third resin cartridge containing dipentyl pentylphosphonate; and collecting Lu-177 having passed through the third resin cartridge onto a third collection column having resin containing DGA.

### CROSS-REFERENCE TO RELATED APPLICATIONS

This application claims priority from U.S. Provisional Pat. Application No. 63/253,333 filed on Oct. 7, 2021, in the United States Patent and Trademark Office. The disclosure of which is incorporated herein by reference in its entirety.

### FIELD OF THE INVENTION

The present invention relates to a Lu-177 radiochemistry system and method.

### BACKGROUND OF THE INVENTION

Targeted radiotherapy treatments for delivering a dose of radiation to diseased cells are desired. Lutetium-177 (commonly referred to as Lu-177 or ¹⁷⁷Lu) is a therapeutic isotope of increasing interest to the nuclear medicine community. There are existing methods that employ Lu-177. The production of Lu-177 via neutron capture starting with enriched Ytterbium-176 (commonly referred to as Yb-176 or ¹⁷⁶Yb) targets is known. See Horowitz, Applied Radiation and Isotopes 63 (2005) 23-36.

However, the present invention overcomes problems and shortcomings with existing methods and is a novel system and method for providing significant improvements to Lu-177 radioisotope product quality and yield as a radiopharmaceutical.

### SUMMARY OF THE INVENTION

The present invention relates to a Lutetium-177 (commonly referred to as Lu-177 or ¹⁷⁷Lu) radioisotope radiochemistry system and method. More specifically, the method of the present invention is directed to a method of making the Lu-177 radioisotope at a yield and purity suitable for pharmaceutical use.

The method of the present invention uses a purified Ytterbium(III) oxide (Yb₂O₃) to produce a high purity Ytterbium (Yb) target material to improve radioisotope product quality. The Lu-177 radioisotope system and method of the present invention seeks to efficiently improve both the radioisotope product quality and yield.

The method of the present invention uses incorporation of real-time spectroscopy, automation, and re-circulation options to help control the separation process and to monitor the degradation of resin(s) used for the separation of or for separating Yb and Lu.

The method of the present invention identifies suitable materials and acid concentrations to accommodate the flow of product through the separation process and storage of the product.

The method of the present invention optionally comprises pretreating or purifying enriched Yb₂O₃; neutron irradiation/capture of the enriched Yb₂O₃ to produce ¹⁷⁷Yb that subsequently decays by β⁻-emission to a combination of ¹⁷⁶Yb¹⁷⁷Yb¹⁷⁷Lu; and retrieval of the enriched Yb₂O₃

The method of the present invention comprises dissolving enriched Yb₂O₃ (preferably with heat using a HNO₃ nitric acid solution) to result in a dissolved enriched Yb₂O₃; processing of the dissolved enriched Yb₂O₃ in a pre-coarse column containing a resin prepared from (2-ethyl-1-hexyl)phosphonic acid mono(2-ethyl-1-hexyl)ester (HEH[EHP]), also referred to herein as an LN2 resin, or an equivalent resin; introducing the dissolved solution onto a chromatographic guard column containing resin prepared from (2-ethyl-1-hexyl)phosphonic acid mono(2-ethyl-1-hexyl)ester (HEH[EHP]), also referred to herein as an LN2 resin, to separate micro amounts of Lu from remaining macro amounts of Yb; passing through a resin cartridge containing dipentyl pentylphosphonate (such as UTEVA® resin commercially available from Eichrom Technologies) for Lu adventitious metals purification (such as Th and U); collecting Lu-177 onto a first guard column having a resin containing tetraoctyl diglycolamide (DGA); washing Lu-177 onto a second column containing LN2 resin; collecting of Lu-177 onto a second DGA guard column; washing Lu-177 onto a third column containing LN2 resin; collecting of Lu-177 onto a third collection column having a resin containing DGA; passing a stream exiting the third DGA guard column through a pre-filter resin/column for trace organics removal; conducting pyrolysis; and reconstituting. The method may further comprise dosing.

In accordance with a feature of the method of the present invention, purifying or processing of enriched Ytterbium(III) oxide (Yb₂O₃) prior to fabrication of irradiation targets and/or irradiation improves specific activity and radionuclidic purity of the final product. Purifying or processing of the enriched Ytterbium(III) oxide (Yb₂O₃) preferably occurs by dissolution of oxide in high purity acid (“high purity” referring to trace metals being typically in the ppb range) and processing through an LN2 resin column capable of separating multi-gram quantities of Yb from Lu, capture of the Yb material onto a resin cartridge and collection column (or alternatively evaporating the Yb dilute HNO₃ solution directly thereby reducing Yb losses), elution using dilute or low molarity HCl, and conversion of chloride eluate into a final oxide form for target irradiation. Lu or other Lanthanide contaminants are removed to improve specific activity and radionuclidic purity of final product. The method is used to remove metallic contaminants and to reduce activation radionuclide impurities to improve overall product quality and mitigate waste costs.

In an aspect of the invention, the method of the present invention uses a column containing a resin bed capable of separating multi-gram quantities of Yb from Lu for processing of dissolved enriched Yb₂O₃. The resin bed is scaled to multi-gram target amounts and batch sizes above approximately 1 Ci Lu-177. Preferably, purification occurs in a separate hot cell from secondary/tertiary processing. The method of the present invention provides for separation of this front-end processing away from the cleaner process steps and mitigation of possible contamination of a secondary processing facility by Yb target powder or contaminants originating from the reactor. This upstream column serves as a “dirtier” pre-coarse column by loading a lot of impurities onto the column. The system provides separate areas where purified product and cleaning can be downstream.

In accordance with an aspect of the present invention, the system and method of the present invention incorporate higher resolution gamma spectroscopy system in-process detectors. The system provides for an automated smarter system. Activity measurement probes are replaced with multi-channel analyzing sensors. Small size solid-state detectors are shielded for localized placement either adjacent or attached to formulation equipment. Gamma peak selective detection allows resolution between Yb isotopes and Lu-177 during separation. The method and system of the present invention allows for more accurate segregation of Yb and Lu-177 during the column passes. Gamma line selectivity provides a more accurate detection of the Yb target isotopes and the Lu-177 to permit more accurate partition of the output to either Lu-177 collection or diversion to Yb capture (or waste).

In accordance with a feature of the present invention, the method of the present invention provides for improved final product Lu yield with use of optimized materials. For example, the amount of Lu-177 that remains adhered to the glassware can be reduced. Target dissolution container material can be selected for low leaching.

In accordance with the present invention, the method of the present invention improves final product dissolution following pyrolysis by incorporating higher normality acid, such as higher than 0.045 N HCl. For example, with initial volume addition of HCl, then a final addition of purified water to the desired normality can better control activity concentration and normality. This feature is used to reduce the amount of Lu-177 that remains adhered to the pyrolysis vessel.

In accordance with a feature of the method of the present invention, final product Lu-177 yield can be improved with an optimized crucible material for pyrolization. The crucible material can be replaced with Pt or Ta or some other low leaching, high temperature resistant material. Purity of the crucible material should improve with each run batch or lot. It may also be useful to use an alternative to the pre-filter to aid in pyrolysis (e.g., charcoal instead of pre-filter resin). This feature is used to reduce the amount of Lu-177 that remains adhered to the pyrolysis vessel.

In accordance with a recirculation/recycle feature of the method of the present invention, secondary and tertiary process steps can be converted into a singular recycling process using one column containing LN2 resin. Recycling is advantageous in case of resin shortage.

Further areas of applicability of the present invention will become apparent from the detailed description provided hereinafter. It should be understood that the detailed description and specific examples, while indicating the preferred embodiments of the invention, are intended for purposes of illustration only and are not intended to limit the scope of the invention.

### BRIEF DESCRIPTION OF THE DRAWINGS

The present invention will become more fully understood from the detailed description and the accompanying drawings, which are not necessarily to scale, wherein:

FIG. 1 is a block diagram illustrating a process in a Lu-177 radiochemistry system in accordance with an aspect of the present invention.

FIG. 2 illustrates a flow path for obtaining purified enriched Yb₂O₃ for preparing reactor targets in accordance with an embodiment of the present invention.

FIG. 3 illustrates a coarse separation flow path as part of the process in accordance with an aspect of the present invention.

FIG. 4 illustrates a fine separation flow path with a recirculation option as part of the process in accordance with an aspect of the present invention.

FIG. 5 illustrates a single column recirculation option with reuse of a recirculating fine column in accordance with an aspect of the present invention.

FIG. 6 illustrates a two column recirculation option in accordance with an aspect of the present invention.

### DESCRIPTION OF THE PREFERRED EMBODIMENTS

The following description of the embodiments of the present invention is merely exemplary in nature and is in no way intended to limit the invention, its application, or uses. The following description is provided herein solely by way of example for purposes of providing an enabling disclosure of the invention, but does not limit the scope or substance of the invention.

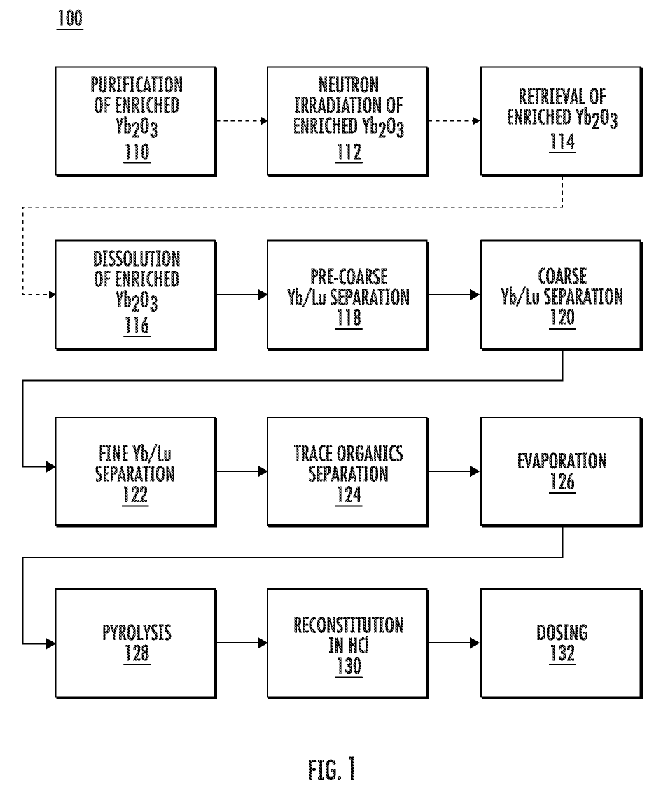

Referring to the figures, FIG. 1 is a block diagram illustrating a process in a Lu-177 radiochemistry system in accordance with an aspect of the present invention. As shown in the block diagram of FIG. 1, process 100 of the present invention generally comprises: purification of an enriched Yb₂O₃ (step 110); neutron irradiation/capture of the enriched Yb₂O₃ (step 112); and retrieval of the enriched Yb₂O₃ (step 114).

Since steps 110, 112, and 114 occur prior to dissolution and one could conduct the process beginning with purification step 110. Alternatively, one could obtain an enriched Yb₂O₃ as a starting material. In which case, the process comprises: dissolving enriched Yb₂O₃ (step 116); pre-coarse Yb/Lu separation (step 118); coarse Yb/Lu separation (step 120); fine Yb/Lu separation (step 122); trace organics separation (step 124); evaporation (step 126); pyrolysis (step 128); reconstitution in HCl (step 130); and dosing (step 132). Preferably, Yb₂O₃ is dissolved in 0.5 N to 2 N HNO₃ at a temperature of 150° C. to 250° C.

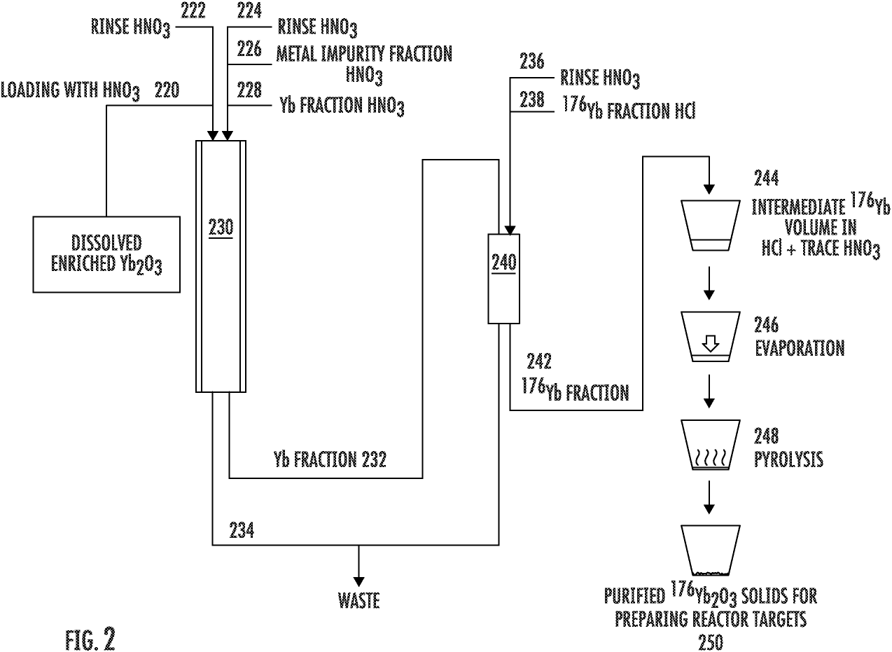

FIG. 2 illustrates a flow path for obtaining purified enriched Yb₂O₃ for preparing reactor targets in accordance with an embodiment of the present invention. In FIG. 2, dissolved enriched Yb₂O₃ is loaded (shown at 220) with 0.001 N to 0.1 N HNO₃ onto a pre-coarse column 230 containing a resin prepared from (2-ethyl-1-hexyl)phosphonic acid mono(2-ethyl-1-hexyl)ester (HEH[EHP]), also referred to as an LN2 resin, or containing an equivalent resin. Pre-coarse column 230 has approximately ≤ 100 cm³ bed volume (B.V.). An amount of the dissolved enriched Yb₂O₃ exits pre-coarse column 230 in an exit stream 234 and goes to waste.

Loading is followed by a rinse with 0.01 N to 0.5 N HNO₃ (shown at 222) entering pre-coarse column 230, and an amount of rinse 222 exits pre-coarse column 230 in exit stream 234 and goes to waste.

This is followed by a rinse with 0.5 N to 2 N HNO₃ (shown as 224) that produces a combined metal impurity fraction with 0.5 N to 2 N HNO₃ (shown as 226) and an Yb fraction with 0.5 N to 2 N HNO₃ (shown as 228) which as a function of time pass through pre-coarse column 230. Respective amounts of 224, 226, and 228 exit pre-coarse column 230 and go to waste.

An exiting stream 232 with Yb fraction exits pre-coarse column 230 and goes to a column 240 containing a resin containing tetraoctyl diglycolamide (DGA). Column 240 is also referred to herein as an Yb column 240.

A rinse of 0.01 N to 0.5 N HNO₃ (shown at 236) enters column 240, and a ¹⁷⁶Yb Fraction with 0.01 N to 0.5 N HCl (shown at 238) also enters column 240. An exiting stream 242 containing a ¹⁷⁶Yb fraction exits column 240 and goes to an intermediate ¹⁷⁶Yb volume (shown at 244) in 0.01 N to 0.5 N HCl with trace HNO₃ and then to evaporation (shown at 246) at 95° C. to 250° C., then to pyrolysis (shown at 258) at 500° C. to 800° C., and then to purified ¹⁷⁶Yb₂O₃ solids (shown at 250) for preparing reactor targets. The waste from 228 and from 236 are appropriately designated as being separate from waste from 220, 222, 224 and 226, which is non-usable waste.

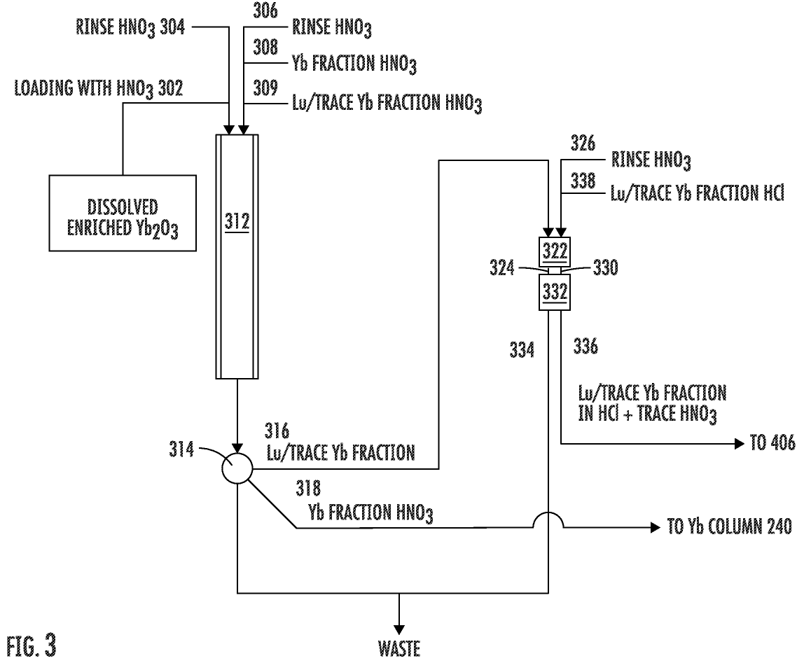

FIG. 3 illustrates a coarse separation flow path as part of the process in accordance with an aspect of the present invention. In FIG. 3, dissolved enriched Yb₂O₃ is loaded (shown at 302) with 0.001 N to 0.5 N HNO₃ onto a chromatographic guard column 312, also referred to herein as coarse column 312, containing a resin prepared from (2-ethyl-1-hexyl)phosphonic acid mono(2-ethyl-1-hexyl)ester (HEH[EHP]), also referred to as an LN2 resin, or containing an equivalent resin. Coarse column 312 has about 29 cm³ to about 68 cm³ B.V.

This is followed by a rinse (shown at 304) with 0.01 N to 0.5 N HNO₃ that goes to waste. This is followed by a rinse (shown at 306) with 0.5 N to 2 N HNO₃ that produces a combined Yb fraction (shown at 308) with 0.5 N to 2 N HNO₃ and a Lu/trace Yb fraction (shown at 309) with 2 N to 6 N HNO₃ passing as a function of time through coarse column 312.

At a junction of exiting flows from coarse column 312 is a valve 314 controlled by a gamma spectroscopy detector. The exiting Yb fraction (shown at 318) with 0.5 N to 2 N HNO₃ goes to Yb column 240. The exiting Lu/trace Yb fraction (shown at 316) passes through a resin cartridge 322 containing dipentyl pentylphosphonate (such as UTEVA® resin commercially available from Eichrom Technologies) for Lu adventitious metals purification (such as Th and U); and a first stream 324 exiting resin cartridge 322 goes through a guard column 332 having a resin containing tetraoctyl diglycolamide (DGA).

A rinse (shown at 326) with 0.01 N to 0.5 N HNO₃ and a Lu/trace Yb fraction (shown at 328) with 0.01 N to 0.5 N HCl passes through resin cartridge 322 and a second stream 330 exiting resin cartridge 322 goes through guard column 332. A Lu/trace Yb fraction (shown at 336) in 0.01 N to 0.5 N HCl and trace HNO₃ exits from guard column 332 and goes to a recirculating fine column 406. An exiting waste stream (shown as 334) from guard column 332 resulting from 316 and 324 go into waste.

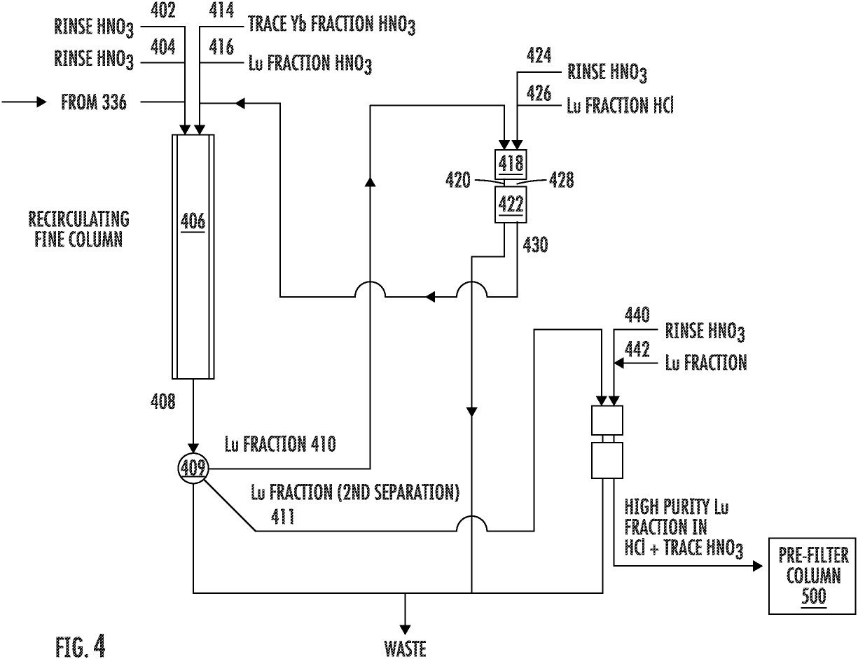

FIG. 4 illustrates a fine separation flow path with a recirculation option as part of the process in accordance with an aspect of the present invention. In FIG. 4, the flow containing Lu/trace Yb fraction in 0.01 N to 0.5 N HCl and trace HNO₃ (shown at 336) combines with a rinse (shown at 402) with 0.5 N to 2 N HNO₃ and a rinse (shown at 404) with 0.01 N to 0.5 N HNO₃ and loads onto a chromatographic guard column 406. The chromatographic guard column 406, also referred to herein as recirculating fine column 406, contains a resin prepared from (2-ethyl-1-hexyl)phosphonic acid mono(2-ethyl-1-hexyl)ester (HEH[EHP]), also referred to as an LN2 resin, or containing an equivalent resin. Recirculating fine column 406 has a B.V. in a range of about 29 cm³ to about 68 cm³.

A flow (shown as 414) containing a trace Yb fraction with 0.5 N to 2 N HNO₃ combines with a Lu fraction with 2 N to 6 N HNO₃, and the combined flow enters recirculating fine column 406.

A flow (shown as 408) exits recirculating fine column 406 and enters a valve 409 that is controlled by a gamma spectroscopy detector. Valve 409 divides the exiting flow/stream 408 into 3 flow paths: a Lu fraction (shown as 410) that passes through a resin cartridge 418 containing dipentyl pentylphosphonate (such as UTEVA® resin commercially available from Eichrom Technologies). An exiting stream (shown as 420) from resin cartridge 418 passes through a guard column 422 having a resin containing tetraoctyl diglycolamide (DGA).

A Lu fraction (second separation) (shown as 411) passes through to a resin cartridge 432 containing dipentyl pentylphosphonate (such as UTEVA® resin commercially available from Eichrom Technologies). An exiting stream (shown as 434) from resin cartridge 432 passes through a guard column 436 having a resin containing tetraoctyl diglycolamide (DGA).

A valve waste stream (shown as 412) resulting from 312, 404, 402, 414, and 416 goes to waste.

Lu fraction 410 goes to resin cartridge 418 containing dipentyl pentylphosphonate (such as UTEVA® resin commercially available from Eichrom Technologies). Exiting stream (shown as 420) from resin cartridge 418 passes through guard column 422 having a resin containing tetraoctyl diglycolamide (DGA). An exiting stream (shown as 430) from guard column 422 passes through recirculating fine column 406. A resulting waste stream (shown at 438) exits along with other waste streams resulting from 424, 426 and 440 to Waste.

A rinse (shown at 424) with 0.01 N to 0.5 N HNO₃ and a Lu fraction (shown at 426) with 0.01 N to 0.5 N HCl passes through resin cartridge 418 containing dipentyl pentylphosphonate (such as UTEVA® resin commercially available from Eichrom Technologies). Exiting stream (shown as 420) from resin cartridge 418 passes through guard column 422 having a resin containing tetraoctyl diglycolamide (DGA).

A rinse (shown at 440) with 0.01 N to 0.5 N HNO₃ and a Lu fraction (shown at 442) with 0.01 N to 0.5 N HCl passes through resin cartridge 432 containing dipentyl pentylphosphonate (such as UTEVA® resin commercially available from Eichrom Technologies). Exiting stream (shown as 444) from resin cartridge 432 passes through guard column 436 having a resin containing tetraoctyl diglycolamide (DGA). An exiting stream (shown as 446) containing a high purity Lu fraction in 0.01 N to 0.5 N HCl and trace HNO₃ goes to a pre-filter column.

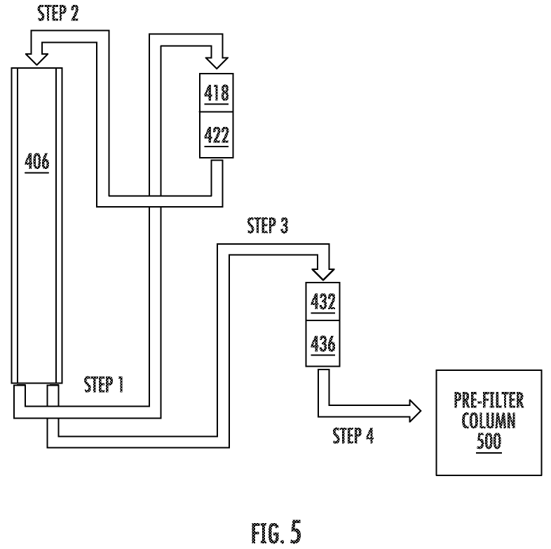

FIG. 5 illustrates a single column recirculation option with reuse of recirculating fine column 406. Recirculating fine column 406 is sized between 29 cm³ and 68 cm³ B.V. Process flow uses existing stages of a column containing a resin prepared from (2-ethyl-1-hexyl)phosphonic acid mono(2-ethyl-1-hexyl)ester (HEH[EHP]), referred to herein as an LN2 resin, or an equivalent resin and a collection column having resin containing tetraoctyl diglycolamide (DGA) such that eluate from DGA is re-directed back into the same column containing LN2 resin, or an equivalent resin. Separate DGA columns are maintained to ensure optimal Lu-177 capture and product purity. Primary separation would be handled by dedicated Yb target processing column having LN2 resin, or an equivalent resin. Referring to FIG. 5, a first stream exits from recirculating fine column 406 and into resin cartridge 418 containing dipentyl pentylphosphonate (such as UTEVA® resin commercially available from Eichrom Technologies). An exiting stream from resin cartridge 418 passes through a guard column 422 having a resin containing tetraoctyl diglycolamide (DGA). An exiting stream from guard column 422 passes through recirculating fine column 406. A second stream exits from recirculating fine column 406 and into resin cartridge 432 containing dipentyl pentylphosphonate (such as UTEVA® resin commercially available from Eichrom Technologies). An exiting stream from resin cartridge 432 passes through a guard column 436 having a resin containing tetraoctyl diglycolamide (DGA). Exiting stream from guard column 436 proceeds to a pre-filter column.

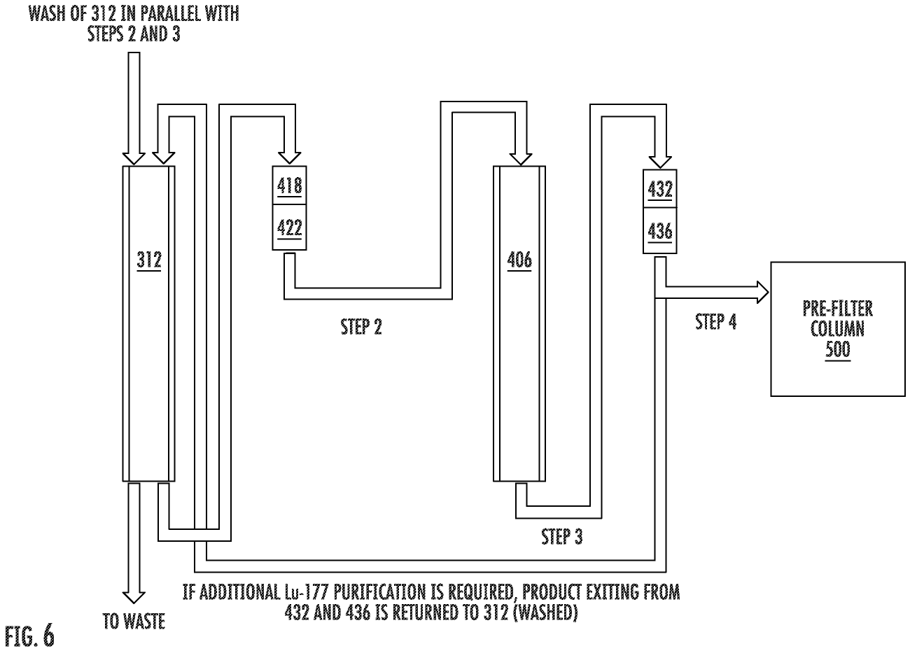

FIG. 6 illustrates a two column recirculation option in accordance with an aspect of the present invention. Referring to FIG. 6, a two stage system can be used where the coarse column 312 is being rinsed at the same time recirculating fine column 406 is being loaded, so the first one can be re-used if needed. Provides for closed-loop flow. Referring to FIG. 6, wash enters coarse column 312 in parallel with steps 2 and 3 as shown. A first stream exiting coarse column 312 goes to waste. A second stream exiting coarse column 312 passes through resin cartridge 418 containing dipentyl pentylphosphonate (such as UTEVA® resin commercially available from Eichrom Technologies) and into guard column 422 having a resin containing tetraoctyl diglycolamide (DGA). In step 2, the stream exiting guard column 422 and passes through recirculating fine column 406. In step 3, the stream exiting recirculating fine column 406 passes through resin cartridge 432 containing dipentyl pentylphosphonate (such as UTEVA® resin commercially available from Eichrom Technologies) and into guard column 436 having a resin containing tetraoctyl diglycolamide (DGA). If additional Lu-177 purification is required, the exiting stream from resin cartridge 432 and guard column 436 is returned to coarse column 312 (washed). After additional Lu-177 purification is no longer required, the exiting stream from resin cartridge 432 and guard column 436 is directed to a pre-filter column as step 4.

There are numerous features of the system and method of the present invention that are advantageous including but not limited to the following. Pre-treatment of the Yb₂O₃ to produce a higher purity Yb target material is used to improve radioisotope product quality. The use and size as well as the flow rates for and paths through the various resin modules reduce the separation process time which effectively increases yield. Incorporation of real-time spectroscopy, automation, re-circulation options to help control of the separation process and to monitor the degradation of the resin materials.

It will therefore be readily understood by those persons skilled in the art that the present invention is susceptible of broad utility and application. Many embodiments and adaptations of the present invention, other than those herein described, as well as many variations, modifications and equivalent arrangements, will be apparent from or reasonably suggested by the present invention and the foregoing description thereof, without departing from the substance or scope of the present invention. Accordingly, while the present invention has been described herein in detail in relation to its preferred embodiment, it is to be understood that this disclosure is only illustrative and exemplary of the present invention and is made merely for purposes of providing a full and enabling disclosure of the invention. The foregoing disclosure is not intended or to be construed to limit the present invention or otherwise to exclude any such other embodiments, adaptations, variations, modifications, and equivalent arrangements.

What is claimed is:

1. A method of making Lu-177, the method comprising:
dissolving an enriched Yb₂O₃,
optionally processing the dissolved enriched Yb₂O₃ in a pre-coarse column containing resin prepared from (2-ethyl-1-hexyl)phosphonic acid mono(2-ethyl-1-hexyl)ester (HEH[EHP]);
loading the dissolved enriched Yb₂O₃ on a first chromatographic guard column containing resin prepared from (2-ethyl-1-hexyl)phosphonic acid mono(2-ethyl-1-hexyl)ester (HEH[EHP]) to separate Lu-177 from Yb;
passing a first separation of a stream exiting from the first chromatographic guard column through a first resin cartridge containing dipentyl pentylphosphonate;
collecting Lu-177 having passed through the first resin cartridge containing dipentyl pentylphosphonate onto a first collection column having resin containing tetraoctyl diglycolamide (DGA);
loading an exiting stream from the first collection column having resin containing DGA on a second chromatographic guard column containing resin prepared from (2-ethyl-1-hexyl)phosphonic acid mono(2-ethyl-1-hexyl)ester (HEH[EHP]) to separate Lu-177 from Yb;
passing a first separation of a stream exiting from the second chromatographic guard column through a second resin cartridge containing dipentyl pentylphosphonate;
collecting Lu-177 having passed through the second resin cartridge containing dipentyl pentylphosphonate onto a second collection column having resin containing tetraoctyl diglycolamide (DGA);
passing a second separation of a stream exiting from the second chromatographic guard column through a third resin cartridge containing dipentyl pentylphosphonate; and
collecting Lu-177 having passed through the third resin cartridge containing dipentyl pentylphosphonate onto a third collection column having resin containing tetraoctyl diglycolamide (DGA).

2. The method according to claim 1, further comprising passing a stream exiting from the third collection column having resin containing DGA through a pre-filter column for trace organics removal.

3. The method according to claim 1, further comprising evaporating.

4. The method according to claim 1, further comprising conducting pyrolysis.

5. The method according to claim 1, further comprising reconstituting.

6. The method according to claim 1, further comprising conducting neutron irradiation of enriched Yb₂O₃ prior to dissolution.

7. The method according to claim 6, further comprising retrieval of the enriched Yb₂O₃ prior to dissolution.

8. The method according to claim 6, further comprising purifying the enriched Yb₂O₃.

9. The method according to claim 1, wherein the dissolved enriched Yb₂O₃ is loaded onto the first chromatographic guard column at a temperature of 150° C. to 250° C.

10. The method according to claim 1, wherein the dissolved enriched Yb₂O₃ is loaded on the first chromatographic guard column with 0.001 N to 0.5 N HNO₃.

11. The method according to claim 1, wherein the first chromatographic guard column has a bed volume of about 29 cm³ to about 68 cm³.

12. The method according to claim 10, wherein loading is followed by a first rinse with HNO₃.

13. The method according to claim 12, wherein the first rinse with HNO₃ is in a range of 0.01 N to 0.5 N HNO₃.

14. The method according to claim 12, wherein the first rinse is followed by a second rinse with HNO₃.

15. The method according to claim 14, wherein the second rinse with HNO₃ is in a range of 0.5 N to 2 N HNO₃.

16. The method according to claim 14, wherein the second rinse produces a combined Yb fraction with 0.5 N to 2 N HNO₃ and a Lu/trace Yb fraction with 2 N to 6 N HNO₃ passing into the first chromatographic guard column.

17. The method according to claim 16, wherein the Lu/trace fraction passes in the first separation of the stream exiting from the first chromatographic guard column through the first resin cartridge containing dipentyl pentylphosphonate and onto the first collection column having resin containing tetraoctyl diglycolamide (DGA).

18. The method according to claim 1, wherein the exiting stream from the first collection column contains a Lu/trace fraction in HCl and trace HNO₃.

19. The method according to claim 1, wherein the second chromatographic guard column has a bed volume in a range of about 29 cm³ to about 68 cm³.

20. The method according to claim 1, wherein the exiting stream from the first collection column having resin containing DGA combines with a first rinse with 0.5 N to 2 N HNO₃ and a second rinse with 0.01 N to 0.5 N HNO₃ prior to loading on the second chromatographic guard column.

21. The method according to claim 1, wherein a flow containing a trace Yb fraction with 0.5 N to 2 N HNO₃ combines with a Lu fraction with 2 N to 6 N HNO₃, and the combined flow enters the second chromatographic guard column.

22. The method according to claim 1, wherein the stream exiting the second chromatographic guard column enters a valve separating the exiting flow into at least the first separation and the second separation.

23. The method according to claim 22, wherein the valve is controlled by a gamma spectroscopy detector.

24. The method according to claim 1, wherein a rinse with 0.01 N to 0.5 N HNO₃ and a Lu fraction with 0.01 N to 0.5 N HCl is added to the second resin cartridge containing dipentyl pentylphosphonate.

25. The method according to claim 1, wherein the stream exiting the second collection column having resin containing tetraoctyl diglycolamide (DGA) enters the second chromatographic guard column.

26. The method according to claim 1, wherein a rinse with 0.01 N to 0.5 N HNO₃ and a Lu fraction with 0.01 N to 0.5 N HCl is added to the third resin cartridge containing dipentyl pentylphosphonate.

27. The method according to claim 1, wherein a stream containing a high purity Lu fraction in 0.01 N to 0.5 N HCl and trace HNO₃ exits from the third collection column having resin containing tetraoctyl diglycolamide (DGA).

28. The method according to claim 27, wherein the stream containing the high purity Lu fraction in 0.01 N to 0.5 N HCl and trace HNO₃ goes to a pre-filter column.

29. The method according to claim 1, further comprising recirculation with at least one chromatographic guard column containing resin prepared from (2-ethyl-1-hexyl)phosphonic acid mono(2-ethyl-1-hexyl)ester (HEH[EHP]).
